(19) United States
(12) Patent Application Publication
(10) Pub. No.: US 2003/0220578 A1
(43) Pub. Date: Nov. 27, 2003

(54) Cardiac gating method and system

(76) Inventors: Vincent B. Ho, N. Bethesda, MD (US); Mark C. Haigney, Annapolis, MD (US)

Correspondence Address: MORRISON & FOERSTER LLP, 1650 TYSONS BOULEVARD, SUITE 300, MCLEAN, VA, 22102, US

(21) Appl. No.: 10/285,702

(22) Filed: Nov. 1, 2002

Related U.S. Application Data
(60) Provisional application No. 60/330,894, filed on Nov. 2, 2001.

(51) Int. Cl. A61B 5/04

(52) U.S. Cl. 600/521

(57) ABSTRACT

The invention provides new materials and devices for EKG gating and defibrillation that alleviate problems in the art. Embodiments of the invention utilize special electrodes of certain dimensions and made from materials that can generate trigger signals or transmit pulses more reliably and/or with less interference to other diagnostic procedures. The electrodes and systems improve the amplitude and overall reliability of detecting EKG signals. The improved signals enable more reliable detection of a true cardiac phase and improved cardiac gating. Thus, embodiments of the invention lead to improved image quality, more accurate imaging of cardiac and intrathoracic/upper abdominal structures and improved referencing of systemic arterial blood flow for blood flow measurement within the chest and elsewhere in the body, including, for example, the extremities.

### REFERENCE TO RELATED APPLICATIONS

The application claims priority to U.S. Provisional Application entitled “Cardiac Gating Method and System” assigned Application No. 60/330,894, and filed Nov. 2, 2001, the entirety of which is specifically and entirely incorporated by reference.

### BACKGROUND

1. Field of the Invention

The invention relates generally to medical diagnostics, cardiac resuscitation and more particularly to electrodes used for cardiac care and diagnosis.

2. Background of the Invention

Medical resonance imaging (“MRI”) is very useful for diagnosing medical conditions. MRI can help evaluate heart disease by generating images of structures such as a beating heart. However, movement of such structures during an MRI study can result in significant blurring of their images and other non-diagnostic information. This movement problem is particularly troublesome for MR imaging of the heart and its surrounding structures because of the physiologic motion of the heart over the cardiac cycle. The indiscriminate acquisition of MR data from differing phases of the cardiac cycle for an image thus often results in poorly resolved images of the heart [1].

A solution to the poor resolution problem has been to synchronize MRI signal acquisition by “gating” the MR data acquisition with the cardiac contraction cycle as described by Lanzer et al. in Radiology 150:121-127 (1984) and Lanzer et al. in Radiology 155:681-686 (1985). The gating technique significantly improves the quality of resultant images and has become very popular. Cardiac-gating enables MRI data acquisition during a specific cardiac phase [2-7]. Imaging during diastole, for example, minimizes blurring artifacts related to cardiac contraction because the heart is relatively less active then. The ability to image properly during a specific cardiac phase enables the identification of abnormalities that are cardiac phase-specific (e.g. valvular insufficiency). Cardiac gating also allows appropriate referencing of images obtained over the cardiac cycle (throughout systole and diastole) in a “cine” mode. Cine MR imaging is central for illustration and/or measurement of cardiac blood flow as well as vascular blood flow in general. Furthermore, the ability to accurately perform cardiac-gated data acquisitions enables the achievement of not only sharper image detail but also higher spatial resolution images.

Cardiac gating typically utilizes electrocardiogram (EKG) information obtained from three or more small electrodes (FIG. 1) placed on the anterior chest for supine patient imaging or upper back (FIG. 2) for prone patient imaging. The EKG electrodes may be 1-2 inches in diameter and include self-adhesive edges and central gel portions to enhance EKG signal conduction. The electrodes, their leads and associated EKG equipment typically have been fashioned after those used for routine clinical diagnostic EKG.

A typical EKG-gating procedure for MRI entails placing several (typically 3 or 4) small 1-2 inch electrode discs on the skin surface. Leads from electrodes placed on the patient's skin connect to either a separate gating device or the actual MR scanner. All materials used for the EKG, including leads to electrodes must be MRI compatible. That is, the materials should not generate excessive heat or significantly affect the magnetic field leading to distortion of the MR image. In particular, the metal used in EKG electrodes, particularly paramagnetic and more particularly ferromagnetic materials cause problems in the MRI environment and often are used sparingly.

EKG derived gating is useful for improving MRI and for other diagnostic procedures that need a temporal cardiac reference. The EKG signal typically is filtered and augmented to allow a software algorithm implemented in a computer to identify the R wave as the “trigger” or main reference point for the procedure. Recently, Chia et al (J Magn Reson Imaging 2000;12:678-688) described a more sophisticated ECG vector analysis algorithm for proper R wave detection [7]. The augmentation of the native MRI signal would also improve the overall efficiency in these algorithms for proper R wave detection.

While contributing to improved performance of diagnostic procedures, EKG-gating has certain problems, which result in deteriorated signals, particularly when used with MRI. One problem is that EKG gating during MR imaging often leads to a phenomenon called “T wave elevation” when patients are in the MR scanner [8,9]. If the R wave signal is poor, the elevated T wave may be mistaken for the R wave and erroneously trigger image acquisition. The increased signal, or T wave “swell” may be identified incorrectly as an R wave, which triggers imaging at the opposite time period than that intended. As a result of the backwards timing, the image may be obtained during the systole period instead of the diastole period or vice versa.

Another problem is a poor EKG signal that may be inherently weak or a result from the patient's body habitus geometry precluding good EKG signal reception at the skin surface. A poor signal may induce improper gating because the gating circuitry and/or software used for switching cannot determine as reliably when to take a diagnostic reading. Yet another problem is that the electrical signal generator (the heart) has an unoptimized axis (e.g. rightward axis) that is difficult to capture spatially even with standard electrode placement. Still another problem is that the electrical coupling between an electrode lead and a patient's skin degrades over time due to perspiration and/or movement during the examination period.

The distortion problem in an MRI scan from metal electrodes and conductors was addressed by van Genderingen et al., who showed that graphite materials need to be reinforced with plastic to prevent breakage [10]. This group used electrodes that were similar in size and placement to regular EKG electrodes. In this context, others have stressed the need, if not absolute requirement to use at least 12 electrodes, as commented on by Burch et al. [11] and Melendiz et al. [12]. Thus, much of the work in this field has addressed the problem of electrode breakage and using enough electrodes to obtain desired details concerning localization of EKG signal which can have significant impact on cardiac diagnosis.

A related problem of electrode use around MRI equipment is that electrodes designed for defibrillators generally interfere with MRI diagnoses and are unsuited for use during MRI procedures. In fact, present defibrillators generally are strictly used outside of the MRI environment because of the danger that these electrodes pose, primarily due to their metal composition, to MRI. Unfortunately, as a result of this, cardiac defibrillation, as well as other forms of cardiopulmonary resuscitation (CPR) are deemed incompatible within an MR scanner. Should an emergency arise during an MRI exam, the patient is generally removed from the actual scanner room and the feature of an un-locking scanner table is standard for an MRI scanner. CPR if required is performed outside the scanner room. Thus the requisite equipment for CPR (i.e. defibrillator equipment and large patches) generally has not been designed for use in the MR scanner room [13]. A patient is placed at risk during MRI because of the extra delay in carrying out an electrical resuscitation at a separate location, when avoiding this problem.

These limitations of present equipment have serious consequences. Poor EKG signal processing during MRI can in some cases prevent the detection of serious arrhythmias, thereby leading to delay in appropriate therapy. Furthermore, the EKG signals are susceptible to the generation of artifactual electrical signals. This interference may hinder technicians who operate the equipment from properly identifying life-threatening electrical problems until after a cardiac arrest event. Successful defibrillation is extremely time-sensitive because mortality increases by approximately 10% with each minute of delay until administration of an appropriate counter shock. Thus, poor EKG monitoring during MRI can significantly degrade safety of the diagnostic test.

These problems persist despite incremental improvements to EKG gating for MRI measurements [1-7]. Many such improvements concern proper patient preparation, such as the use of abrasive gels prior to lead placement; advances in small electrode design, typically by using improved materials to improve EKG signal reception; improved lead design, using cables that connect electrodes to EKG recording/filtering devices and/or to the MR scanner itself; and improved detection algorithms to identify the R wave [2-7]. Most of these improvements are modifications to existing hardware and software developed for regular EKG monitoring. Thus, most of the technology in this field is based on the needs of standard EKG assay and does not address as well the problems of EKG triggering, particularly in the MRI environment. For example, ferrous and other paramagnetic materials often are used for electrodes and connectors. These materials interfere with the faithful generation of an MRI signal and actually prevent the use of certain equipment such as defibrillation electrodes near MRI equipment.

### SUMMARY OF THE INVENTION

The invention provides new materials and devices for EKG gating and defibrillation that alleviate problems in the art. Embodiments of the invention utilize special electrodes of certain dimensions and made from materials that can generate trigger signals or transmit pulses more reliably and/or with less interference to other diagnostic procedures. The electrodes and systems improve the amplitude and overall reliability of detecting EKG signals. The improved signals enable more reliable detection of a true cardiac phase and improved cardiac gating. Thus, embodiments of the invention lead to improved image quality, more accurate imaging of cardiac and intrathoracic/upper abdominal structures and improved referencing of systemic arterial blood flow for blood flow measurement within the chest and elsewhere in the body, including, for example, the extremities.

One embodiment of the invention directed to defibrillation allows cardiac resuscitation in an MRI facility itself, thus minimizing patient movement. In an advantageous embodiment, electrodes used for detecting EKG signals as described herein are used for a second function of defibrillation.

These electrodes and systems for their use particularly are useful for EKG triggering in combination with diagnostic systems that often interfere with regular EKG electrode measurements such as MRI. The invention also is useful for cardiac synchronization of other imaging or diagnostic studies such as cardiac nuclear scintigraphy (e.g. stress thalium, stress sestimibi, etc.), computed tomography (CT, electron beam CT, multi-detector CT, etc.), computed tomography angiography and stress echocardiography.

Embodiments of the invention also embrace the synchronization of image-guided therapies as required for radiation or other ablative therapies where sensitive regions (e.g. beating heart) are sometimes avoided or included within the procedural field. Such therapies include, for example, superconducting open configurations for image guided therapy as described by Schenck et al. [14], tumor ablation as described by Cline et al. [15], microwave thermal ablation as described by Chen et al. [16] and radiofrequency endocardial ablation using real time three dimensional magnetic navigation as described by Shpun et al. [17]. Results of such therapies may be monitored by MRI to determine anatomic changes and even temperature changes from the therapy. In each case, proper EKG gating facilitates proper timing for the cardiac therapy either by ensuring proper or improved imaging of, for example, the catheter (i.e. higher detail may be required to see catheter or target structure), potentially augmenting the therapy or simply enabling proper selective timing of ablation. In addition, the invention would also improve EKG monitoring of therapeutic progress and results for certain cardiac interventions (e.g. cardiac ablative therapies for arrhythmias). An embodiment of the invention is a system for prospective and retrospective cardiac gating in a medical procedure for diagnosis of a patient comprising at least two large area electrodes, wherein each electrode has a minimum skin surface contact area of two square inches. In a related embodiment each electrode has a minimum skin surface contact area of at least three square inches. In other embodiments each electrode has a minimum skin surface contact area of at least four square inches and larger.

Another embodiment of the invention comprises an electrocardiogram electrode having a minimum surface contact area of 4 square inches and a resistance across its largest diameter of at least 10 ohms.

Another embodiment of the invention comprises a system for cardiac gating in a medical procedure used for diagnosis of a patient comprising at least two large area electrodes, wherein each electrode has a minimum skin surface contact area of 4 square inches and a signal processing unit for determining cardiac phase, wherein each electrode is electrically connected to the signal processing unit and the signal processing unit outputs a signal in response to an R wave as a reference point for imaging.

Another embodiment of the invention comprises an electrocardiogram electrode that has a minimum surface area of 4 square inches and that comprises an organic conductor.

Other embodiments and advantages of the invention are set forth, in part, in the following description and, in part, may be obvious from this description, or may be learned from the practice of the invention

### DESCRIPTION OF THE FIGURES

FIG. 1 depicts anterior electrode placement using conventional EKG electrodes and large electrode patches of the invention.

FIG. 2 depicts posterior electrode placement using conventional EKG electrodes and large electrode patches of the invention.

FIG. 3 shows EKG signal results obtained using large electrode patches (top graph) and conventional small electrodes (bottom graph).

FIG. 4 shows the effect of voluntary patient movement on EKG signals obtained using large electrode patches (top graph) and conventional small electrodes (bottom graph).

FIG. 5 shows the effect of patient respiration on EKG signals obtained using large electrode patches (top graph) and conventional small electrodes (bottom graph).

### DESCRIPTION OF THE INVENTION

The inventors rejected the paradigm of others in this field who evolved EKG triggering electrodes by either using conventional EKG electrodes or by making incremental changes to EKG equipment. It was surprisingly discovered that greater reliability signals needed for EKG triggering can be obtained by trading on some ability to generate highly detailed information on local EKG signals. Thus, embodiments of the invention use large surface skin electrodes that, while less suited for some detailed EKG recording are more robust and superior for triggering. The large surface area desired in many embodiments was found to work very well for detecting gross EKG detail suitable for timing while being less sensitive to small changes in electrode positioning, bulk patient movement or internal visceral movements (e.g. respiratory motion), due to the greater surface area involved. The larger surface area also enabled improved skin contact during exercise and stress induced imaging whereby significant changes in patient or visceral positions may occur and subject perspiration may facilitate electrode displacement during the exam. The larger surface area also improved safety by minimizing interaction with electromagnetic fields such as that used for MRI, thereby providing less chance of tissue bums to the patient.

The larger surface area further improved patient safety by providing more reliable heart rate and rhythm information due to improved reliability of EKG measurement, especially cardiac R waves, with a greater signal to noise ratio. In embodiments, the signal to noise ratio is 25%, 50%, 100%, 200%, and even 500% higher compared to that obtainable with regular electrodes and systems as described, for example, in Example 1 [18]. The improvements in signal to noise ratios are seen particularly with normal hospital patients, who often are sweaty and nervous, and breath rapidly and generally irregularly. Comparison measurements are taken as averages from 10 typical cardiovascular patients who exhibit one or more of these symptoms.

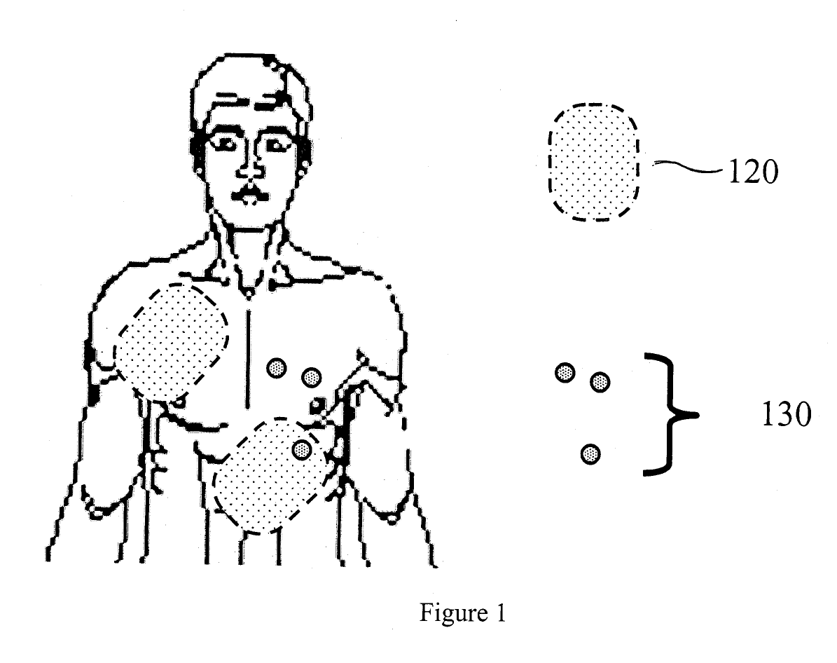

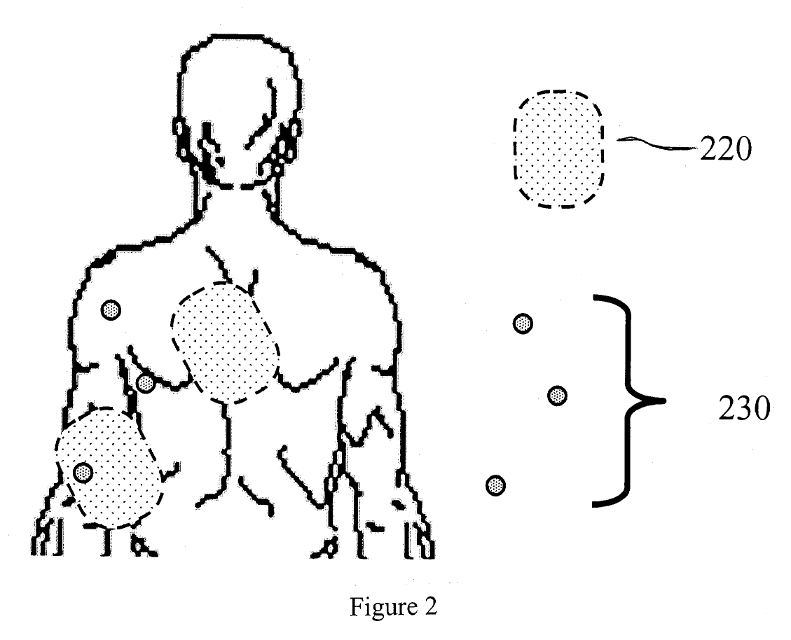

The inventors further discovered that they could achieve accurate EKG triggering with only two or three electrodes. In embodiments of the invention, two or more large self-adhesive electrode patches 220, as shown in FIG. 2, are placed over the chest and/or upper back to detect broad detail of an EKG signal suitable for triggering. Two large electrodes may be placed at different locations as can be readily determined by a medical practitioner to optimize generation of a difference electrical signal suitable for triggering. In one embodiment an electrode is placed on the right shoulder or midline of the right chest and another electrode is placed below the left nipple at or lateral to the anteroaxillary line. Conventional small electrodes 130 and 230, are shown in FIGS. 1 and 2 respectively. In an embodiment small electrode(s) are used in combination with large electrode(s). One or more electrodes for body ground optionally are used as an electrical reference point.

It was further surprisingly discovered that a meshwork of minimal-metallic conductor material is less sensitive to the formation of eddy currents induced by a changing electromagnetic field and is more compatible with techniques such as MRI. Yet another discovery was that making the electric connection between an electrode and its lead more diffuse, particularly in combination with high impedance materials provides a more stable signal for EKG triggering. In preferred embodiments the mesh size is at least 10 times smaller than the wavelength of the electromagnetic radiation. In a more preferred embodiment the mesh size is at least 100 times smaller and in a yet more preferred embodiment the mesh size is at least 1000 times smaller.

Electrodes and their leads, according to embodiments of the invention are less sensitive to magnetic fields produced by equipment such as MRI instrumentation. In particular, the materials disclosed herein and objects made from them generally may be used in the presence of magnetic fields that exceed 5 Gauss. These materials may be employed in other systems as well that include one or more electric circuits and which are useful during MRI. A large number of medically useful devices are known that rely on electrical circuits and which may be improved by use of the materials and devices taught herein.

High Electrode Surface Area

Electrodes of preferred embodiments have large surface areas. This feature minimizes electrical noise and other vagaries due to body movement and local changes in conductivity due to perspiration and the like. Preferred embodiments of the invention utilize two or three high surface area electrodes. By “high surface area” electrodes is meant electrodes that have larger surface areas in contact with a patient's skin compared with most electrodes that are used for EKG recording. In these embodiments the term “high surface area” means more than 4 square inches, preferably more than 6 square inches, yet more preferably more than 8 square inches, even more preferably more than 10 square inches, yet still more preferably more than 12 square inches, yet more preferably more than 16 square inches and most preferably, for use with adults, more than 20 square inches and even more preferably 24 square inches.

A desirable result from this embodiment is that signals picked up by the electrodes electronically are normalized (e.g. averaged) to the entire electrode surface before the signal is sent to a detector. This property improves the signal quality by normalizing out deviations over small electrical skin contact areas that otherwise may create electric noise in smaller electrodes. This feature trades off the ability to interrogate local EKG signals obtained with more conventional electrodes with a more reliable recording of global EKG signal that reveals basic timing details.

Improved CPR Electrodes According to the Invention

If CPR is necessary when a patient is undergoing MRI diagnosis, the patient typically is removed from the MR scanner room (and magnetic field) by unlatching the scanner table from its docking position in the scanner room. CPR then is administered outside the MR scanner room. These acts incur extra delay in responding to heart fibrillation. An embodiment of the invention addresses this problem by providing a CPR electrode that is made substantially from minimal-metallic material and which is compatible with MRI and other like procedures.

The conducting material of a CPR electrode according to this embodiment of the invention comprises a substantially minimal-metallic material that preferably is in a mesh or fabric configuration to minimize interaction with electromagnetic fields. Preferably, the electrode mesh or fabric has an interweave or mesh size (spacing between adjacent conducting fibers that form a space in electrode) that is at least 10 times smaller than the mean wavelength of the electromagnetic signal, more preferably at least 100 times smaller and even more preferably at least 1000 times smaller than the wavelength to inhibit absorption of electromagnetic energy and thereby alleviate interference with the diagnostic assay. Of course, a suitable weave can be chosen by routine experimentation to allow penetration of the electromagnetic signal, while minimizing formation of eddy currents in the electrode from absorption of electromagnetic radiation. Preferably the weave of the material is transparent to at least some MRI signal. The phrase “is transparent to at least some MRI signal” means that less than 90 percent of an MRI signal is absorbed by passage through the material. In an embodiment less than 50% of an MRI signal is absorbed by the material and in another embodiment less than 20% is absorbed. Preferred electrodes will be radiolucent, that is, having the property of not blocking ionizing radiation for CT and nuclear medicine applications.

A preferred conducting material for the electrode of this embodiment is carbon prepared from a polymer such as a polyacetylene, poly(p-phenylene), poly(p-phenylene vinylene), poly(p-phenylene sulfide), polyacetylene, polyanaline, polyquinoline, polypyrrole, and/or polythiophene. Generally, an organic electrode material such as a carbon polymer based material is manufactured by an oxidizing process that converts a starting polymer(s) into a higher carbon content substance. Accordingly, one embodiment of the invention is a process for making an EKG electrode or defibrillation electrode, comprising the steps of: providing an organic carbon polymer such as a polymer selected from the above list in the form of a strand, cloth or fiber particle; oxidizing the strand, cloth or fiber particle to increase the carbon context, preferably by heating; and shaping (typically by spinning, weaving and cutting) the oxidized strand, cloth or fiber into a two dimensional form suitable for contacting the skin of a patient. In a preferred embodiment the material is processed by a first heating step in the presence of at least 10 percent oxygen, which oxidizes the material, followed by a second heating step carried out in the absence of (i.e. less than 1%, preferably less than 0.1%, and more preferably less than 0.01%) oxygen.

During use of the electrode, the shaped conductive material preferably is combined with an ionic gel to improve electrical contact with the skin. Ionic gels developed for use near MRI equipment are well known and contemplated for this use. The preferred mesh-like form provides greater surface area to an ionic gel, thus allowing lower resistance. When used as material for a defibrillation electrode, the electrical resistance of the prepared material at 20 degrees centigrade preferably is less than 100 ohms per square meter, more preferably less than 10 ohms per square meter, still more preferably less than 5 ohms per square meter and, for some applications yet more preferably less than 2 ohms per square meter. When used as material for an EKG triggering electrode or other EKG electrode, the electrical resistance of the prepared material at 20 degrees centigrade preferably is less than 1000 ohms per square meter, more preferably less than 100 ohms per square meter, and still more preferably less than 10 ohms per square meter. In other embodiments where impedance of the EKG input circuitry is suitably high the preferred electrodes may have electrical resistance of greater than 1,000 ohms per square meter or even greater than 10,000 ohms per square meter.

Electrode Composition

Electrodes may be made from a variety of conducting materials and combinations of materials. For use during magnetic resonance imaging or another procedure that generates high electromagnetic radiation in the presence of MRI, electrodes preferably lack appreciable (i.e. contain less than 10% by weight of total conducting material, not including contact gel) paramagnetic material. More preferably the electrode material has less than 5% paramagnetic material, yet more preferably less than 1% paramagnetic material and most preferably less than 0.1% of the total conductivity as paramagnetic material, in order to avoid interfering with the MRI signal. In another embodiment the electrodes lack appreciable (i.e. less than 10% by weight of total conductive material not including contact gel) metal, preferably less than 1% and more preferably less than 0.1% by weight metal.

In a desirable embodiment the conductor in an electrode contains “minimal metallic” composition. The term “minimal metallic” means that the material is less than 25% by weight metallic. More preferably the material has less than 10% metallic material, yet more preferably less than 5% metallic material and most preferably less than 1% of the total weight as metallic material.

In another embodiment the conductor in an electrode is at least 75% by weight organic material. In another embodiment the organic conductor comprises at least 90% of the conducting material and in another embodiment the organic conductor comprises at least 99% of the conducting material, by weight. The terms “organic material” and “organic conductor” as used here refer to material that includes at least 90% by weight elements selected from the following group: carbon, sulfur, oxygen, silicon, germanium, hydrogen, nitrogen, phosphorous, and selenium. More preferably the organic material comprises at least 95% by weight one or more of the listed elements. In a preferred embodiment the organic conducting material is more than 95% by weight carbon.

In another embodiment of the invention a metallic conductor is used in combination with minimal-metallic conductor(s) or non-conductors as mechanical support. When used in combination with MRI, however, it is most preferred to avoid paramagnetic material as conductor for the electrode and for the electrode lead.

Conducting Polymers

In a particularly preferred embodiment of the invention the conducting material within an electrode and/or its contacting lead comprises (preferably at least 75% by weight, more preferably at least 90%, yet more preferably at least 99%) conducting polymer(s). Conducting organic materials are especially preferred for use with MRI and other techniques because such materials are less susceptible to interference with the electromagnetic energy produced by these procedures.

It was surprisingly discovered that combining a minimal-metallic conductor material with a high surface area electrode is particularly useful for EKG and EKG triggering systems and can alleviate problems such as induced electric (eddy) currents in the electrodes and their leads, which can even lead to burning in very extreme cases. Without wishing to be bound by any one theory of this embodiment of the invention, it is believed that the organic conductor material inherently is resistant to induced (eddy) currents and generally is transparent to radio waves, particularly when the electrode comprises a mesh rather than a solid surface. The conductivity of the organic conductor can be adjusted to provide a desired high impedance to MRI radiation induced current yet the conductor may be prepared in a thick enough mat to provide ample conductivity of the EKG signal. It was discovered or realized that a thick organic polymer in the form of a mesh is superior to metallic electrodes because the organic electrode is opaque to MRI, generates very little eddy current, yet can be connected by a single lead to a large area surface contact of the body. In an embodiment, an electrode made from an organic conductor or other conductor responds to an MRI signal with a low temperature increase rate and is safer than typical small electrodes used previously.

Conductive polymers have been studied intensively for more than twenty years and a large variety are known. Initially polyacetylene, a conjugated organic polymer was reported as having high electric conductivity when oxidized by suitable reagents. The concept of conductivity and electroactivity of conjugated polymers was quickly broadened from polyacetylene to include a number of conjugated hydrocarbon and aromatic heterocyclic polymers, such as poly(p-phenylene), poly(p-phenylene vinylene), poly(p-phenylene sulfide), polyacetylene, polyanaline, polyquinoline, polypyrrole, and polythiophene, while success with fluorocarbon polymers was reported more recently as described in U.S. Pat. No. 6,208,075. The principal methods for preparing conducting polymers have included electrochemical oxidation of resonance-stabilized aromatic molecules, structure modification along with doping, and synthesis of conducting transition metal-containing polymers. Each of these materials alone, in combination and also combined with metallic conductors may be used in embodiments of the invention for both the electrodes and/or electrode leads.

A particularly useful embodiment of the invention relies on carbonized fabric made from polyacrylonitrile or other substance for the electrode material. Carbonized fabric that is electrically conductive and suitable for making large area electrodes is known to the skilled artisan as, for example taught in U.S. Pat. No. 6,172,344 to Gordon et al., which is incorporated by reference in its entirety, particularly the lower half of column 6, which describes how to synthesize a fabric of organic conductor. This patent describes the heating/oxidation of polyacrylonitrile fiber. The treated fiber contains a “virtual 100% carbon content,” was finished in a fabric form of 270 gm/square meter weight and exhibited an electrical resistance at 20 degrees centigrade in the range 3-4.5 ohms per square meter across the width and 1.5 to 2.5 ohms per square meter along the length. The conductive fabric can be encapsulated or laminated on one side with any of a range of materials, as for example, described on column 9 and Table 1 of U.S. Pat. No. 6,172,344, which is particularly incorporated by reference.

Electrode Leads

In one embodiment of the invention that differs from conventional EKG electrodes, a lead may be attached to a large electrode over a large surface or edge region of that electrode for greater conductivity and to normalize the EKG response over the entire electrode pad. Such diffused contact between electrode and its lead were discovered to allow use of more MRI resistant material for the lead and for the junction between the lead and electrode while maintaining higher conductivity. In one embodiment the electrode is an organic polymer as described above and the lead is connected diffusedly throughout a region of the electrode. In another embodiment the lead is attached along an edge of at least 0.25 inches, more preferably 0.5 inches and yet more preferably at least 0.75 inches long. In another embodiment the lead is attached over a surface area of at least 0.5 square inches and more preferably at least 1 square inch.

In an embodiment for use with MRI, a lead to an electrode can have a substantial portion (i.e. less than 5%) of its total conductive mass made from non-paramagnetic material(s). The use of paramagnetic materials preferably is avoided in an MRI environment because a paramagnetic material may interact with an MRI field and degrade the detected signal. In an embodiment at least 75% conductivity of the electrode arises from the organic polymer(s) or oxidized polymer(s). Many organic polymers conduct less well than metals such as iron, copper and silver. However an organic polymer may be manufactured as a thick piece, having a low (less than 1,000 ohms, preferably less than 100 ohms, more preferably less than 10 ohms, yet more preferably less than 5 ohms) electrical resistance per square meter. In one embodiment the electrode and the lead to the electrode are made mostly (at least 75%, more preferably at least 90% by weight) of the same material and the lead forms a cable extending from the surface of the electrode.

The use of a thick minimal-metallic conductor is useful for MRI because it allows the use of a high impedance material in a manner that provides good conductivity between the skin surface and measuring equipment. Without wishing to be bound by any one theory for this embodiment of the invention, the high impedance also helps reduce electrode heating. This embodiment as well as other embodiments of the invention provide electrodes that heat up with a low temperature increase rate, that is, an increase in temperature of the electrode mass caused by radio frequency and magnetic field variation typical of exposure in clinical MRI scanners. The term “low temperature increase rate” means that the electrode mass temperature increase is less than a conventional electrode such as that described in Example 1, when used for gating during an MRI exam with a 1.5 Tesla scanner. For example, a desirable electrode will increase in temperature less than 50%, less than 25%, or even less than 10% than the typical small electrode described in Example 1 and described more fully by Jerzewski and Wall [18].

In an embodiment of the invention, a single electrode pad may have multiple leads on it. Each lead may correspond to a separate small conductivity electrode region within the pad, and the signals from the alternative electrode-lead combinations may be averaged out in the connected equipment by hardware or software. Alternatively, each lead may connect to a different region of a large area conducting electrode. In the latter case, optionally the large area electrode has a high enough impedance such that most of the current flowing through the attached lead will be associated (sourced or sinked) at the electrode surface nearest the lead. In an embodiment a large number of leads are connected electrically to different locations on a large area electrode surface of relatively high impedance. In this way, a higher impedance is used while minimizing lead impedance for maximum resistance to radio frequency field effects. Of course, a “single electrode pad” that has “multiple leads on it” also includes a large area conducting electrode having separate regions that are electrically isolated and that have a separate lead attached to each region.

Electrode Systems for EKG Triggering

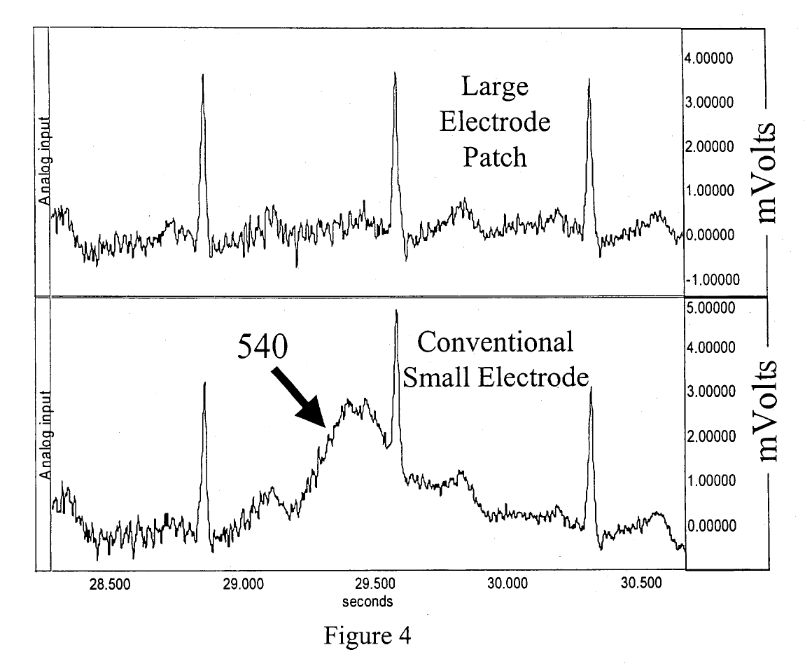

The electrodes, through their leads, are coupled to EKG triggering systems for use with diagnostic procedures such as MRI. Such systems can be used directly or after some modification, in embodiments of the invention. Generally, a system will have at least two electrodes and an optional ground to generate a signal that is resolved by circuitry and a computer program to generate a signal that corresponds to step in the cardiac cycle. The optional ground can be anywhere but preferably on the upper torso. FIGS. 7-4 from Jerzewski and Wall shows representative placements of a ground electrode, cardiovascular magnetic resonance electrodes, and ECG electrodes. These placements may be used for electrode and/or ground placements when measuring the effects of MRI on electrode temperature and signal to noise measurements.

The hardware input circuit can be thought of as seeing three sources of impedance, the impedance of the hardware itself, the impedance of the electrodes and leads, and the impedance of the body tissue that is connected to the electrodes. Preferably the impedance of the hardware is greater than the combined impedance of the electrodes, leads and body tissue. More preferably the hardware impedance is at least 10 times greater and even more preferably the hardware impedance is at least 100 times greater than the combined impedance of the electrodes and leads. Embodiments of the invention provide lower impedance for the body tissue by virtue of the greater surface area compared to the surface area of EKG electrodes used by others. In preferred embodiments conductive polymers are used that give (electrode plus lead) impedances that are less than 10%, more preferably less than 1% and yet more preferably less than 0.1% of the input impedance of the electronics. Shielding and cabling materials and systems known in the art may be used in many embodiments, as well as hardware that has already been developed for EKG triggering and are not described here further.

In one embodiment of the invention, the external circuitry (leads, any electronic filters such as low pass filters, electrodes, body tissue) time constant is adjusted to reject high frequency pulses such as high frequency eddy currents and higher frequency components of an EKG signal that is not needed for triggering. In another embodiment the EKG triggering hardware and/or software is adjusted to reject one or more of these high frequency components. In one aspect, embodiments of the invention provide an inherently low frequency discriminating system, by virtue of the larger area pads (which have higher capacitances) that pick up slower moving pulses more preferably than the faster moving pulses. These embodiments also pick up body movements less readily, which often have higher frequency components.

The advantageous features of desirable electrodes reviewed here have significant consequences for improved safety. For example, it was found that the large area electrodes generally can provide more reliable R wave information compared to the small electrode systems used previously. This feature allows more reliable monitoring of heart rate and gives greater safety to the overall medical procedure.

Turning now more specifically to the figures, representative embodiments of the invention will be further explained. The following examples are offered to illustrate embodiments of the present invention, but should not be viewed as limiting the scope of the invention.

### EXAMPLES

### Example 1

A conventional MR electrode was compared with a large area patch electrode for recording EKG signals from a human subject. A BIOPAC digital data acquisition system was used for receiving and analyzing the data. The leads of two defibrillation patches (Agilent Technologies, Catalog No. M3501A, Multifunction Adult Defib Electrodes) were stripped and the bare wires inserted into the inputs of the BIOPAK EKG preamplifier. The signal obtained was compared with that recorded simultaneously from two MR conventional electrodes on the same electrical axis as the patches.

Surface EKG signals were recorded from a volunteer during quiet breathing, breath holding, and slow moving of the volunteer to simulate the conditions expected during a typical MR study.

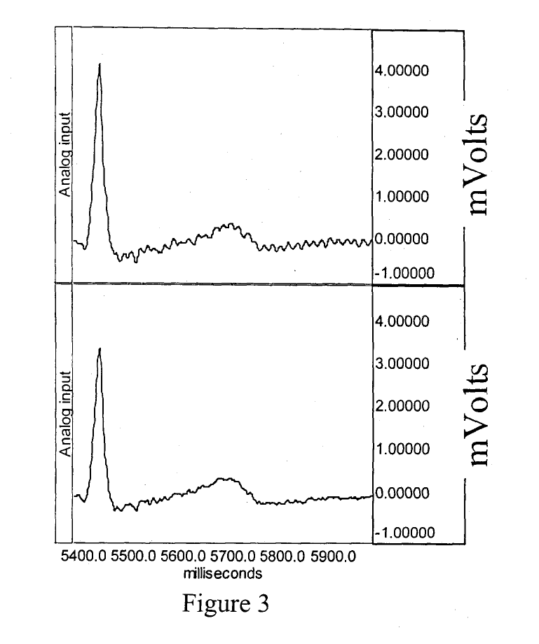

The results showed that the signal recorded from the large area patch was identical in morphology to that from the conventional lead, but was 15-25% greater in amplitude. See FIG. 3, which shows the same scale for both tracings. The top plot of FIG. 3 shows results from the large electrode patch and the bottom plot shows results from use of conventional small electrodes. The increased signal was not affected by changing the preamplifier.

When the human subject moved from side to side a significant artifact, mound 540 was induced easily as seen in the recording in FIG. 4 from the conventional electrodes, but not from the large surface patch. The top plot shows results from the large electrode patch and the bottom plot shows results from use of conventional small electrodes. The noise recorded from the conventional electrodes was sufficiently high enough in amplitude to equal a native electrocardiographic depolarization. That is, the measured noise was strong enough to trigger the MR gating inappropriately. On the other hand, the simultaneous record from the large electrode patch showed no significant artifact, and the two recordings are presented using the same scale. This finding was reproduced.

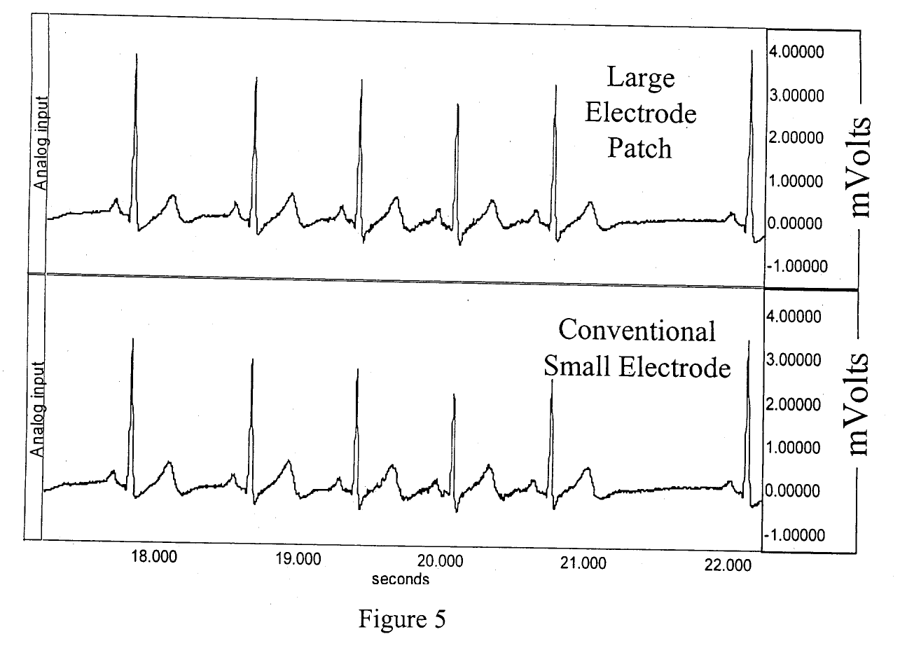

During respiration, diminution was noted in the amplitude of both signals, but the signal from the larger patches was less affected than from the conventional small electrodes as seen in FIG. 5. The top plot shows results from the large electrode patch and the bottom plot shows results from use of conventional small electrodes. The signals from the conventional small electrodes were 29% smaller than those from the large area patch.

### Example 2

Two large area oval shaped electrode patches 7 inches long and 4.25 inches wide are manufactured and used as depicted for EKG gating in FIG. 1. This example used the arbitrary electrode positions indicated in the figure. However, other positions are also contemplated herein. Preferably the two electrodes are not superimposed but have sufficient distance to obtain a signal as will be appreciated by a skilled artisan.

The conductive material of the electrode patch is a carbonized fabric comprising more than 99 percent organic material and is prepared from 1.5 denier polyacrylamide fiber tow as described in U.S. Pat. No. 6,172,344. Briefly, the fiber is continuously baked in an oven at 221 degrees centigrade in the presence of 100 percent oxygen for 10 hours. The fiber is then drawn, spun to 100 fibers in cross section and twisted to 2/14 weight yarn. The yarn is woven into a loose knit fiber having one sixteenth inch interweave spacings. The loose knit material is cut with scissors to form two of the oval shapes. A bundle of fifty, two-foot long fibers are attached to each oval shape by sewing a terminal 1 inch of each with a needle. The oval electrodes with attached bundled leads are baked at 1000 degrees centigrade in the presence of 100 percent nitrogen for fifteen minutes. The product is black and has a resistance of between 2 and 30 ohms per square meter at 20 degrees centigrade.

Each oval conductive textile is assembled into an electrode by adhering an insulating yet stiff plastic layer on the side where the bundle comes off of the oval, with an exit opening for the lead bundle. Regular conductive gel is added to the side opposite the lead attachment site and the ovals are mounted onto a patient as shown in FIG. 1. The leads are electrically connected to an EKG monitor and EKG signals are produced. The oval electrodes are found to produce significantly less interference to MRI measurements compared to that of regular EKG electrodes.

Oval electrodes are made and used for defibrillation as follows. The procedure cited above is used to manufacture an oval cloth, but a thicker bundle of 250 fibers one foot long are attached to each oval to form a lead. Each of the leads is terminated with a friction coupling for attaching a copper wire from a defibrillator. A defibrillator can be used while the patient is in the vicinity of an MRI apparatus after attaching the copper wires to the electrode lead ends. The copper wires are positioned at least a few inches away from the patient at all times. Both the thin lead connected and the thick lead connected carbon oval electrodes are used for obtaining EKG measurements in the presence of an MRI procedure.

Other embodiments and uses of the invention will be apparent to those skilled in the art from consideration of the specification and practice of the invention disclosed herein. All references and other documentary materials cited herein, including all U.S. and foreign patents and patent applications and all priority documents, are specifically and entirely hereby incorporated herein by reference. It is intended that the specification and examples be considered exemplary only, with the true scope and spirit of the invention indicated by the following claims.

1. A system for prospective and retrospective cardiac gating in a medical procedure for diagnosis of a patient comprising at least two large area electrodes, wherein each electrode has a minimum skin surface contact area of three square inches.

2. The system of claim 1, wherein each electrode has a resistance of at least 2 ohms per square meter.

3. The system of claim 2, wherein each electrode has a resistance of at least 10 ohms per square meter.

4. The system of claim 1, wherein each electrode has a lead and wherein the resistance of the lead is at least 100 ohms per 10 cm of length.

5. The system of claim 1, wherein the medical procedure is selected from the group consisting of magnetic resonance imaging, cardiac imaging, cardiac nuclear scintigraphy, computed tomography and echocardiography.

6. The system of claim 1, wherein the electrodes lack paramagnetic material.

7. The system of claim 6, wherein the electrodes lack materials with significant ferromagnetic properties.

8. The system of claim 1 wherein the electrodes are radiolucent.

9. The system of claim 7, wherein the electrodes comprise a conductor that consists of at least 50% carbon and optionally a conductive gel.

10. The system of claim 7, wherein the conductor is at least 95% carbon.

11. The system of claim 1, wherein the electrode conductors comprise a conductive mesh or fabric made from organic material.

12. An electrocardiogram electrode having a minimum surface contact area of 4 square inches and a resistance across its largest diameter of at least 10 ohms.

13. The electrode of claim 12, having a resistance across its largest diameter of at least 100 ohms.

14. The electrode of claim 12, which lacks paramagnetic material.

15. The electrode of claim 14, which lacks materials with significant ferromagnetic properties.

16. The electrode of claim 12, which comprises a conductor that contains at least 50% carbon and optionally a conductive gel.

17. The electrode of claim 16, wherein the conductor comprises at least 90% organic material.

18. The electrode of claim 17, wherein the conductor comprises at least 95% carbon.

19. The electrode of claim 12, wherein the conductor is in the form of a mesh or fabric.

20. A cardiac electrode that can be used within a five Gauss perimeter of an MRI scanner, comprising a conducting organic material and lacking a metallic conductor.

21. The electrode of claim 20, wherein the conducting organic material is in the form of a fabric or mat with a weave that is transparent to at least some MRI signal.

22. A system for cardiac gating in a medical procedure for diagnosis of a patient comprising:
at least two large area electrodes, wherein each electrode comprises a minimum skin surface contact area of 4 square inches; and
a signal processing unit for determining cardiac phase, wherein each electrode is electrically connected to the signal processing unit and the signal processing unit outputs a signal in response to an R wave as a reference point for imaging.

23. The system of claim 22, wherein the medical procedure is selected from the group consisting of magnetic resonance imaging, cardiac imaging, cardiac nuclear scintigraphy, computed tomography and echocardiography.

24. The system of claim 23, wherein the medical procedure is magnetic resonance imaging and the electrodes substantially lack significant ferromagnetic materials.

25. The system of claim 24, wherein the electrodes are electrically connected to the signal processing unit by leads that substantially lack significant ferromagnetic materials.

26. The system of claim 24, wherein each electrode comprises at least 98% non-metallic material by weight and are in a flexible mesh or fabric form.

27. A system for prospective and retrospective cardiac gating in an image-guided intervention medical procedure comprising at least two large area electrodes, wherein each electrode has a minimum skin surface contact area of three square inches.

28. The system of claim 27, wherein the medical procedure is a form of ablative therapy.

29. The system of claim 27, wherein each electrode has a resistance across a largest diameter of at least 2 ohms.

30. The system of claim 27, wherein each electrode has a lead and wherein the resistance of the lead is at least 2 ohms per 10 cm of length.

31. The system of claim 27, wherein the electrodes lack paramagnetic material.

32. The system of claim 27, wherein the electrodes lack materials with significant ferromagnetic properties.

33. The system of claim 27, wherein the conductor is at least 95% carbon.

34. The system of claim 27, wherein the electrode conductors comprise a conductive mesh or fabric made from organic material.

35. A safe system for prospective and retrospective cardiac gating in an MRI procedure for diagnosis of a patient comprising:
at least two large area electrodes, wherein each electrode has a minimum skin surface contact area of three square inches and wherein each electrode responds to a radio frequency and magnetic field variation of a 1.5 Tesla scanner with a low temperature increase rate.

36. The system of claim 35, wherein the low temperature increase rate is less than 25% as high as that obtained from a conventional MR electrode of 1.5 inches diameter.

37. A safe system for prospective and retrospective cardiac gating in an MRI procedure for diagnosis of a patient comprising at least two large area electrodes, wherein each electrode has a minimum skin surface contact area of three square inches and wherein the system detects an R wave signal with a signal to noise ratio that is at least twice as high as that obtained with conventional MR electrodes of 1.5 inch diameter.

38. The system of claim 37, wherein each electrode responds to a radio frequency and magnetic field variation of a 1.5 Tesla scanner with a low temperature increase rate.

39. An imaging system that facilitates earlier detection of cardiac complications during an imaging procedure, comprising the system of claim 1.

40. A system that facilitates therapeutic cardiac defibrillation in patients who encounter cardiac complications during a radiographic study comprising dual function MRI compatible electrodes, wherein a first function is monitoring of at least one EKG signal and a second function is defibrillation.
